(19) United States
(12) Patent Application Publication
(10) Pub. No.: US 2022/0313960 A1
(43) Pub. Date: Oct. 6, 2022

(54) INTRAVENOUS CATHETER APPARATUS

(71) Applicant: Poly Medicure Limited, Faridabad-Haryana (IN)

(72) Inventors: Pankaj Kumar Gupta, Faridabad-Haryana (IN); Girdhari Lal Sharma, Faridabad-Haryana (IN); Ramkumar Sharma, Faridabad-Haryana (IN); Manoj Kumar, Faridabad-Haryana (IN)

(73) Assignee: Poly Medicure Limited, Faridabad-Haryana (IN)

(21) Appl. No.: 17/433,532

(22) PCT Filed: Dec. 21, 2020
(86) PCT No.: PCT/IB2020/062276

§ 371 Date: Aug. 24, 2021

(30) Foreign Application Priority Data
Dec. 24, 2019 (IN) 201911053630

(51) Int. Cl. A61M 25/06; A61M 5/32

(52) U.S. Cl. CPC A61M 25/0618; A61M 25/0606; A61M 5/3291

(57) ABSTRACT

An intravenous catheter apparatus comprising a tubular catheter having a proximal end and a distal end mounted to a catheter hub; a needle defining an axial direction has a wall defining a lumen, a bevel and having a needle shaft with a needle tip at a distal end and a needle hub mounted to the proximal end of the needle shaft; and a needle guard arranged movably on the needle shaft; wherein said needle shaft extends through said tubular catheter such that said needle tip of said needle protrudes from said distal end of said tubular catheter; wherein said needle is provided with an engagement means adapted to engage with said needle guard in order to prevent said needle guard from sliding off said needle tip; wherein said needle has at least one opening having at least one slanted cut.

### FIELD OF THE INVENTION

The invention relates generally to needle devices, systems, and methods used to administer medicine to a patient or to take blood from a patient. More particularly, the present disclosure relates to intravenous (IV) catheter devices or assemblies. In particularly, the present invention relates to the configuration of the opening provided in the needle close to the needle tip, wherein the distance between the needle tip and the opening is arranged such that the opening is covered by a catheter tube when the needle is in the ready position. Using such an opening, the medical practitioner receives immediate feedback whether the venipuncture to the patient was successful by a so-called flashback feature.

### BACKGROUND OF THE INVENTION

An intravenous (IV) catheter apparatus comprising a needle with a needle guard is generally known and, for example, used to administer medicine to a patient or to take blood from a patient. The intravenous (IV) catheter apparatus usually comprises a tubular catheter having a proximal end and a distal end, a needle defining an axial direction and having a needle shaft and a needle tip at a distal end of the needle shaft, wherein the needle shaft extends through the catheter such that the needle tip protrudes from the distal end of the tubular catheter, and wherein the needle shaft comprises an engagement means in the form of change in profile adapted to engage with a needle guard slidably arranged on the needle shaft in order to prevent the needle guard from sliding off the needle tip.

The needle guard serves to prevent a person handling the intravenous catheter apparatus from accidentally coming into contact with the needle tip after placement of the catheter tube in and subsequent removal of the needle from a patient's vein. Thereby, the intravenous catheter apparatus helps to avoid unwanted transmission of blood borne diseases.

In use, the tubular catheter is inserted into a blood vessel of the patient, typically a vein, by means of the needle which will be withdrawn from the catheter after insertion of the tubular catheter into the blood vessel and upon successful venipuncture. When the needle tip enters the blood vessel, blood flows through a lumen of the needle into an opening provided closer to the needle tip at a distal end of the needle, where it can be observed by the person handling the catheter apparatus. The event of blood oozing out of the opening of the needle is called blood flashback and is used by the person handling the catheter apparatus to confirm venipuncture, from which point on the person inserts the catheter as far as desired under a decreased inclination of the needle in order to not puncture the back wall of the blood vessel.

In the case of delayed blood flashback there is a risk that the person handling the catheter apparatus continues to push the needle into the blood vessel under the increased original inclination of the needle until it exits at the rear side of the blood vessel. This second venipuncture represents a risk to the health of the patient as it may result in inner bleeding. Apart from that the catheter apparatus will have to be removed and replaced by a new one, i.e. the process of introducing the catheter will have to be repeated which is unpleasant and painful to the patient.

The profile of the opening provided in the needle in the conventional intravenous catheter apparatus has an issue that the needle guard due to incidental rotation of the needle guard on the needle shaft may get stuck or obstructed by such opening which can impact proper functioning of the device. To explain it further, the needle guards usually contain arms or jaws having ends blocking the needle tip. While being slidably arranged on the needle shaft, incidental rotation of the needle guard on the needle shaft takes place. During such rotations when the opening in the needle shaft and the arms or jaws having needle tip blocking ends of the needle guard are aligned, the needle guard gets obstructed or stuck into the opening impacting the proper functioning of the device. Moreover, such obstructions create drag or friction upon withdrawal of the needle after venipuncture causing pain to the patient.

The profile of the opening provided in the needle in the conventional intravenous catheter apparatus has another issue in that such opening or slots or hole has a size big enough that once the needle is administered through the human body it runs a risk of catching and/or cutting and/or damaging internal tissues in and/or through said opening or slots or hole.

The instant disclsoure intends to overcome these issues.

### OBJECTS OF THE INVENTION

A primary object of the present invention to provide an intravenous catheter apparatus which provides better protection against accidental pricking by the needle tip and which is inexpensive to manufacture.

Another object of the invention is to provide an intravenous (IV) catheter apparatus which allows for safer, convenient and more reliable placement of the catheter in the patient's blood vessel.

Another object of the invention is to provide a needle with at least one opening or hole closer to the tip having a profile which does not cause obstruction for a needle guard upon withdrawal of the needle after venipuncture.

Another object of the invention is to provide a needle with at least one opening or hole closer to the tip having a profile which does not cause obstruction for the arm or arms of a needle guard upon withdrawal of the needle after venipuncture.

Another object of the invention is to provide a needle with at least one opening or hole closer to the tip having a profile which does not cause obstruction for arm or arms having needle tip blocking end of a needle guard upon withdrawal of the needle after venipuncture.

Another object of the invention is to provide at least one opening or hole for the needle closer to the needle tip having at least one slanted cut at an angle (X) increasing in value opposite to the direction of the needle tip towards proximal end of the needle wherein angle (X) of the slant is within the range of 0° to 89°.

Another object of the invention is to provide at least one opening or hole for the needle closer to the needle tip having at least one slanted cut at an angle (X) increasing in value in the direction of the needle tip towards distal end of the needle wherein angle (X) of the slant is within the range of 0° to 89°.

Yet another object of the invention is to provide at least one opening or hole closer to the needle tip formed by two slanted cuts in that one of the cuts has an angle (X) increasing in value in the direction opposite to the needle tip towards proximal end of the needle and the second cut has an angle (Y) increasing in value in the direction of the needle tip towards distal end of the needle wherein the angle (X) and angle (Y) of the slant is within the range of 0° to 89°.

Yet another object of the invention is to provide at least one opening or hole having a size big enough that once the needle is administered through the human body it does not cause any risk of catching and/or cutting and/or damaging internal tissues in and/or through said opening or hole.

### SUMMARY OF THE INVENTION

The intravenous catheter apparatus of the invention comprises a catheter hub arranged at a proximal end of a catheter tube and having an inner surface defining a chamber; a needle defining an axial direction and having a needle tip, wherein the needle extends through the chamber and the catheter tube when in a ready position; a needle guard slidably arranged on the needle and at least partially received in the chamber when the needle is in its ready position, the needle guard including a base portion and first and second arms extending from the base portion, wherein the first arm is deflected radially outwards by the needle against a restoring force when the needle is in its ready position whereby the needle guard is brought into retaining contact with the catheter hub; and retaining means for retaining the needle guard in the chamber as long as the needle tip is received in the needle guard to safely secure or capture the needle tip within the needle guard.

Accordingly one aspect of the present invention relates to an intravenous (IV) catheter apparatus comprising: a tubular catheter having a proximal end and a distal end mounted to a catheter hub; a needle defining an axial direction (A) has a wall defining a lumen, a bevel and having a needle shaft with a needle tip at a distal end and a needle hub mounted to the proximal end of the needle shaft; and a needle guard arranged movably on the needle shaft; wherein said needle shaft extends through said tubular catheter such that said needle tip of said needle protrudes from said distal end of said tubular catheter; wherein said needle is provided with an engagement means adapted to engage with said needle guard in order to prevent said needle guard from sliding off said needle tip; wherein said needle has at least one opening or a hole having at least one slanted cut at an angle.

According to a further aspect of the present invention the needle includes a needle feature close to its needle tip changing the cross-sectional shape of the needle. Such a feature can be an engagement mean in the form of a change in profile i.e. an enlargement of the cross section of the needle, like a surrounding bump or rip formed on the outer circumferential surface of the needle shaft or formed by a local protrusion of added material provided at one particular outer circumferential part of the needle shaft. Alternatively, and easier to manufacture, the needle feature can be formed by a local crimp providing that the needle is locally squeezed and deformed such that it has an oval cross-section which does not fit through the circular opening within the needle guard.

In one of the embodiments, the opening or hole provided on the needle shaft has at least one slanted cut at an angle (X) increasing in value opposite to the direction of the needle tip towards proximal end of the needle. In another embodiment, the opening or hole provided on the needle shaft has at least one slanted cut at an angle (X) increasing in value in the direction of the needle tip towards distal end of the needle. In yet another embodiment, the opening or hole is formed by two slanted cuts in that one of the cuts has an angle (X) increasing in value in the direction opposite to the needle tip towards proximal end of the needle and the second cut has an angle (Y) increasing in value in the direction of the needle tip towards distal end of the needle. The slant angle (X) and angle (Y) with or without radius (R) is within the range of 0° to 89°. The opening has a length (L) and width (W) of varying dimensions.

In a further embodiment, the opening or hole has at least one slanted radial cut wherein the radius (R) of the slanted cut has varying length.

In further embodiments of the invention, one or more opening or hole can be provided on the needle shaft. The functioning the device with one or more opening or hole remains same as other embodiment disclosed herein.

The slanted cut of the opening or hole has a suitable shape, including a shape that is substantially oval, elliptical, square, rectangular, triangular, circular, polygonal, irregular, etc. The opening can be positioned on the needle shaft so as to face in a direction that is substantially similar to, perpendicular to, or otherwise offset from the direction in which the bevel of the needle faces.

The opening or hole has a shape like a perforation forming an opening of a size being enough to allow a blood flashback therethrough. The small size of the opening also assist in not creating any obstruction for the angled end arm of the needle guard upon withdrawal of the needle post a successful venipuncture out of the catheter hub. The small size of the opening also ensure that once the needle is administered through the human body it does not cause any risk of catching and/or cutting and/or damaging internal tissues in and/or through said opening. The opening or hole can be small as compared to needle length e.g. the length or the width of the opening can be less than 10% and/or 20% of the length of the needle.

The opening or hole is provided on the needle shaft closer to the needle tip. The distance between the needle tip and the opening is arranged such that the opening is covered by the catheter tube.

The opening or hole can be provided anywhere on the circumference of the needle shaft in a 360° position except of the bottom of the needle shaft in a direction opposite to the bevel of the needle.

In one of the embodiments, the opening or hole has two sides in that one of the sides is straight and the other side is angled.

The opening or hole can be positioned anywhere from just proximal of the needle tip to just distal of the catheter hub. In one embodiment, the opening is provided in between the engagement mean and the needle tip. In another embodiment, the engagement mean is provided in between the needle tip and the opening.

### BRIEF DESCRIPTION OF THE ACCOMPANYING DRAWINGS

Embodiments of the present disclosure now will be described more fully hereinafter with reference to the accompanying drawings, in which embodiments of the present disclosure are shown. This present disclosure may, however, be embodied in many different forms and should not be construed as limited to the embodiments set forth herein. Rather, these embodiments are provided so that this disclosure will be thorough and complete, and will fully convey the scope of the claims to those skilled in the art. Like numbers refer to like elements throughout. The embodiments of the invention are described in the following description and in the accompanying drawings, wherein:

FIG. 1A illustrates a cross-sectional view of an intravenous catheter apparatus according to the present invention.

FIG. 1B illustrates a detailed view of the encircled portion of FIG. 1A.

FIGS. 2A & 2B illustrate front and side views of a distal part of the needle respectively with an opening as usually used for blood flash back in prior art devices.

FIG. 2C illustrates a detailed view of the encircled portion of FIG. 2B.

FIGS. 3A & 3B illustrate front and side views of a distal part of the needle respectively with an opening which is used for blood flash back according to one embodiment of the present invention.

FIG. 3C illustrates a detailed view of the encircled portion of FIG. 3B.

FIGS. 4A & 4B illustrate side and front views of a distal part of the needle respectively with an opening which is used for blood flash back according to another embodiment of the present invention.

FIGS. 5A & 5B illustrate side and front views of a distal part of the needle respectively with an opening which is used for blood flash back according to further embodiment of the present invention.

FIGS. 6A & 6B illustrate side and front views of a distal part of the needle respectively with an opening which is used for blood flash back according to yet another embodiment of the present invention.

FIGS. 7A & 7B illustrate side and front views of a distal part of the needle respectively with an opening which is used for blood flash back according to yet another embodiment of the present invention.

FIGS. 8A & 8B illustrate front and side views of a distal part of the needle respectively with an opening which is used for blood flash back according to one embodiment of the present invention.

FIG. 8C illustrates a detailed view of the encircled portion of FIG. 8B.

FIGS. 9A & 9B illustrate side and front views of a distal part of the needle respectively with an opening which is used for blood flash back according to another embodiment of the present invention.

FIG. 10A & 10B illustrate front and side views of a distal part of the needle respectively with an opening which is used for blood flash back and covered with a catheter tube attached to a catheter hub according to one embodiment of the present invention.

FIG. 11A & 11B illustrate front and side views of a distal part of the needle respectively with an opening which is used for blood flash back and covered with a catheter tube attached to a catheter hub according to yet another embodiment of the present invention.

FIGS. 12 and 13 illustrate a needle guard of FIG. 1 according to one implementation being slidably arranged and proceeding to protect the needle tip upon withdrawal of the needle after venipuncture according to the present invention.

FIG. 14 illustrates another implementation of the needle guard which can be used with the intravenous catheter apparatus of the present invention.

### DETAILED DESCRIPTION OF THE INVENTION

The embodiments of the present invention relates to an intravenous catheter apparatus which provides better protection against accidental pricking by the needle tip and which is inexpensive to manufacture. While this invention is susceptible of embodiments in many different forms, there will be described herein specific embodiments with the understanding that the present disclosure is to be considered as an exemplification of the principles of the invention and is not intended to limit the invention to the embodiments detailed herein.

As used herein, the term “proximal” refers to a region of the device or a location on the device which is closest to, for example, a clinician using the device. In contrast to this, the term “distal” refers to a region of the device which is farthest from the clinician, for example, the distal region of a needle will be the region of a needle containing the needle tip which is to be inserted e.g. into a patient's vein.

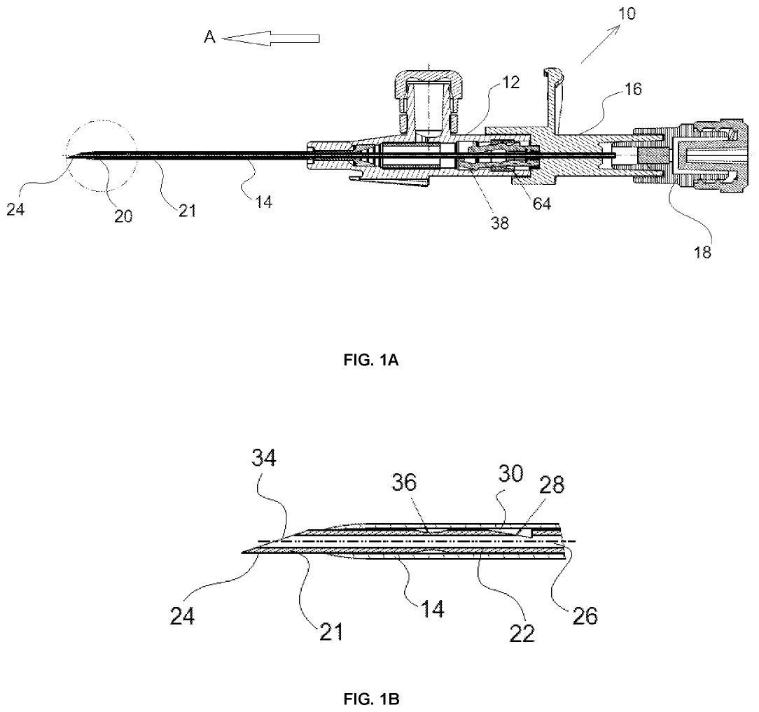

FIG. 1A illustrates by way of an example, a catheter apparatus 10, a needle guard 38 similar to one disclosed in WO2018025094 arranged in a catheter hub 12 near a proximal end 18 of the needle shaft 22 prior to use in a ready position. In this situation, the needle 21 extends completely through the needle guard 38, thereby deflecting the first arm 42 of the needle guard outwards, i.e. at an angle to the axial direction A as can be seen in FIGS. 16 and 17, such that the distal wall or angled end section 56 of the first arm 42 is supported on the needle shaft 22. At least one of the arms 42, 44 have needle tip blocking end, for example an angled end section 56 in one of the embodiments. In another embodiment both the arms 42, 44 of the needle guard 38 can have needle tip blocking ends, for example angled end section 56 at distal ends. Following the insertion of the catheter 14 into a patient, the successful venipuncture is ascertained by the blood visible from an opening or hole 28 provided in the needle 21 closer to the needle tip 24 as illustrated in FIG. 1B. As a result, the intravenous catheter apparatus 10 allows faster and more reliable confirmation of successful venipuncture which makes the handling of the catheter apparatus easier both for the person placing it and the patient on whom it is used. Thereafter, the needle 21 is withdrawn from the catheter tube 14 and the needle shaft 22 moves through the needle guard 38 while the needle guard 38 is retained in the catheter hub 12. Once the needle tip 24 passes the transverse distal wall 56 of the needle guard 38, i.e. such that the needle shaft 22 no longer supports the distal wall 56, a restoring force ensures that the first arm 42 of the needle guard 38 is moved back into alignment with the axial direction A of the needle guard 38, so that the needle tip 24 is blocked by the distal wall 56 of the needle guard 38, i.e. the needle tip 24 is prevented from axially projecting out of the needle guard 38. Thereafter, the needle tip 24 being secured and guarded by the needle guard 38 is pulled out of the catheter hub 12, as a result, the needle guard 38 provides a better protection against accidental pricking and thus increased safety for the person handling the catheter apparatus 10.

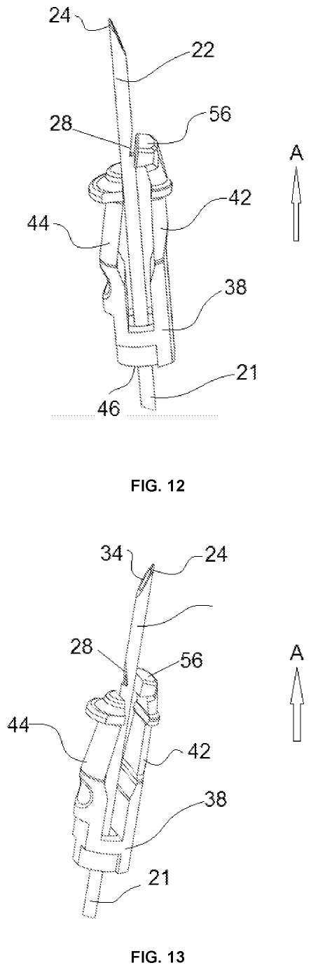

The invention is not limited to only a specific type of needle guard. The invention can be used with various types of needle guards which have needle tip blocking ends for example, as illustrated in FIGS. 12, 13 and 14. Many other implementations are disclosed and contemplated herein. Moreover, it is important to note that the invention is not limited to safety IV catheters, but are applicable to any of a variety of needle products.

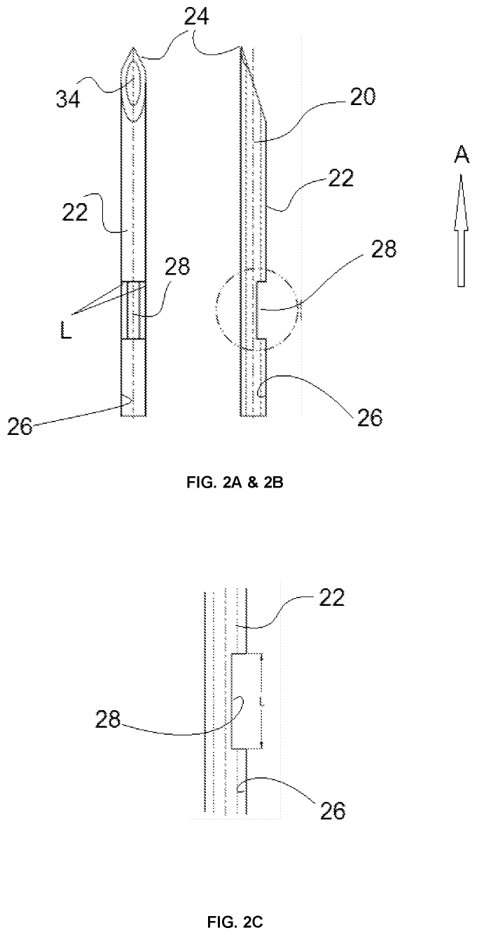

Referring to FIGS. 2A & 2B, front and side views of a distal part of a needle 21 respectively with an opening or hole 28 usually used for blood flash back in prior art devices is shown. FIG. 2C illustrates a detailed view of the encircled portion of FIG. 2B. The opening 28 has a rectangular shape cut having two corners and include length defined by L and width defined by W. The width W is smaller than length L. Referring to FIGS. 16 and 17, it can be seen that the profile of the opening 28 of the needle 21 in FIG. 2C has an issue that the arms of the needle guard 38 may get stuck or obstructed by the corners of such opening 28 due to incidental rotation of the needle guard 38 on needle 21 when the opening 28 and needle tip blocking end, for example an angled end section 56 are aligned. Such obstruction can impact proper functioning of the device 10. Moreover, such obstructions can create drag or friction upon withdrawal of the needle 21 after venipuncture.

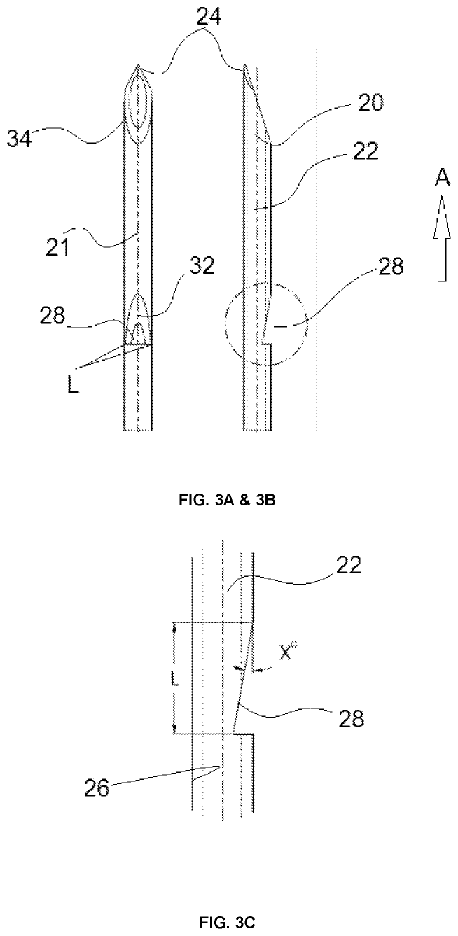

Referring now to FIGS. 3A & 3B, a front and side views of a distal part of the needle 21 respectively with at least one opening or hole 28 which is used for blood flash back according to one embodiment of the present invention is illustrated. The needle 21 having distal 20 and proximal 18 ends extends through a chamber of a catheter hub 12 as well as through the catheter tube 14. In order to improve the visibility of the blood exiting the needle 21 through the opening 28, the catheter tube 14 advantageously comprises a transparent material. A needle hub 16 is attached to the proximal end 18 of the needle 21. As can be seen the opening or hole 28 has two sides in that one of the sides is straight and the other side is angled.

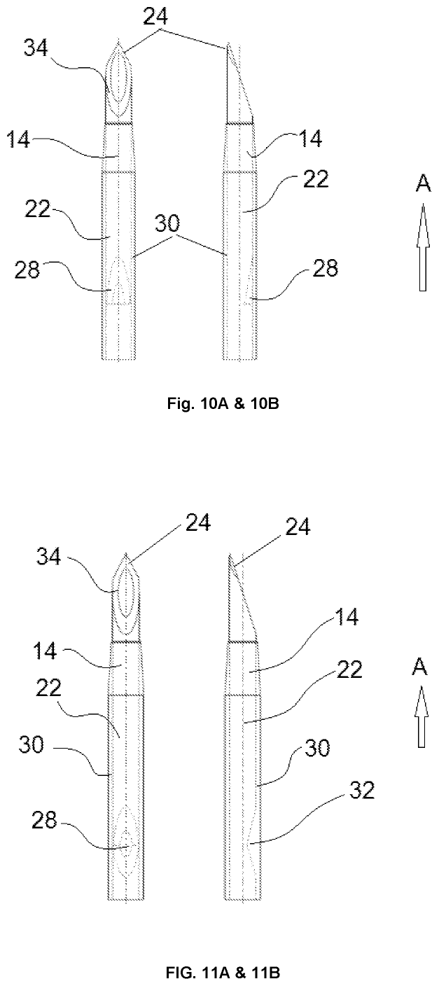

As can be further seen, the needle 21 has a wall defining a lumen 26, a needle shaft 22 and a needle tip 24 at its distal end 20 defining a longitudinal axial direction (A). The needle 21 includes an opening 28 close to the needle tip 24 for quick blood flash back, wherein the distance between the needle tip 24 and the opening 28 is arranged such that the opening 28 is covered by a catheter tube 14 which is attached to a catheter hub 12 when the needle 21 is in the ready position as illustrated in FIGS. 1A, 1B, 10A & 10B and 11A & 11B.

In order to make the insertion of the catheter tube 14 into a patient's blood vessel even more smooth and convenient, the catheter tube 14 is slightly tapered in its distal end 20 region such that the distal end 20 of the catheter tube 14 tightly surrounds the needle shaft 22. To this extent, the distal portion of the catheter tube 14 is tapered inwardly forming an opening that has a size smaller than an outer diameter of the needle 21 which forms a seal around the opening of the catheter tube 14 with the needle 21 to prevent fluid from entering a space 30 between the catheter tube 14 and the needle 21 when the needle tip 24 pierces the skin of a patient. To this end, it is to be understood that the outer diameter of the needle shaft 22 is slightly smaller than the inner diameter of the catheter tube 14 in order to provide a small gap creating space 30 between the needle shaft 22 and the catheter tube 14.

The opening 28 is positioned proximal to the needle tip 24 to the extent such that at least a portion of the opening 28 or the entire opening 28 is not inserted into the patient during venipuncture. The position of the opening 28 is chosen such that it is visible to the user while doing venipuncutre. Thus, the opening 28 can be positioned anywhere from just proximal of the needle tip 24 to just distal of the catheter hub 12.

Due to the opening 28, the medical practitioner receives immediate feedback whether the application to the patient was successful by a so-called flashback feature. The flashback feature provides that a small amount of blood pours out of the opening 28 into the transparent catheter tube 14 upon venipuncture such that it becomes immediately visible in a space 30 between the catheter tube 14 and the needle 21 whether the medical practitioner was successful in introducing the needle tip 24 into the vein of a patient. The blood entering the space 30 between the needle shaft 22 and the catheter tube 14 spreads in the space 30 due to capillary action and can be seen through the transparent material of the catheter tube 14, thereby allowing prompt confirmation of successful venipuncture.

As can be seen, the opening or hole 28 has at least one slanted cut 32 at an angle increasing in value opposite to the direction of the needle tip 24 towards proximal end 18 of the needle 21. The slant angle with or without radius defined by X in FIG. 3C can vary and be within the range of 0° to 89°. The opening 28 has a length defined by L and width W. The dimension of L and W may vary. The shape of the slanted cut 32 of the opening 28 can be of any suitable shape, including a shape that is substantially oval, elliptical, square, rectangular, triangular, circular, polygonal, irregular, etc. For instance, FIG. 3C illustrates an embodiment in which the slanted cut 32 of the opening 28 is substantially oval.

The tip 24 of the needle 21 forms a bevel 34. The opening 28 is typically positioned on a side of the needle shaft 22 such that the bevel 34 of the needle 21 at the needle tip 24 is seen to be in a face up position. However, the position of the opening 28 may vary with respect to the face of the bevel 34 of the needle 21. The opening 28 can be positioned in sides of the needle shaft 22 as well. Thus, the opening 28 can be positioned on the needle shaft 22 so as to face in a direction that is substantially similar to, perpendicular to, or otherwise offset from the direction in which the bevel 34 of the needle 21 faces. Thus, the opening or hole 28 can be provided anywhere on the circumference of the needle shaft 22 in a 360° position except of the bottom of the needle shaft 22 in a direction opposite to the bevel 34 of the needle 21.

To this extent, it is to be understood that each component of the needle 21 may have any suitable characteristic which is necessary for an intrdoucer needle in an intravenous catheter apparatus 10. For example, the distal tip 24 of the needle 21 may comprise a standard bevel, a short bevel, a true short bevel, an angled bevel, a bias grind point, a vet point, a lancet point, a deflected point (anti-coring), or another suitable known or novel needle point. Additionally, the lumen 26 and elongated tubular shaft 22 may be any suitable size necessary an intravenous catheter apparatus 10. For example, the needle 21 may be of any suitable length or any suitable gauge that allows it to be used as the introducer needle in an IV assembly.

The slanted angle of the opening 28 smoothens the passage of the arms 42, 44 of the needle guard 38 being slidably arranged and proceeding to protect the needle tip 24 upon withdrawal of the needle 21 after venipuncture as shown in FIGS. 16 & 17. The slanted angle of the opening 28, thus, does not create an obstruction even when the needle guard 38 rotates on the needle 21 and when the opening 28 and the arms of the needle guard 38 are aligned in the passage of the needle guard 38 from ready to protected position.

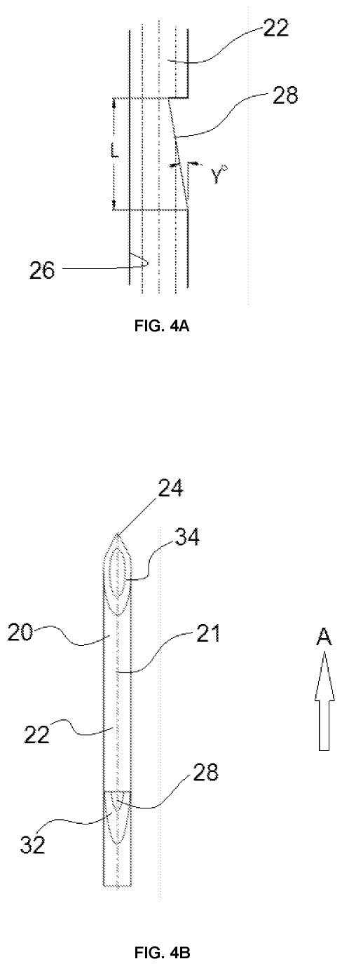

Referring to FIGS. 4A & 4B, they illustrate side and front views of a distal part of the needle 21 respectively with at least one opening or hole 28 which is used for blood flash back according to another embodiment of the present invention. This embodiment differs from the previous embodiment in that the opening 28 has at least one slanted cut 32 at an angle increasing in value in the direction of the needle tip 24 towards distal end 20 of the needle 21. The slant angle with or without radius R defined by X in FIG. 4A can vary and be within the range of 0° to 89°. The opening has a length defined by L and width defined by W each having varying dimensions.

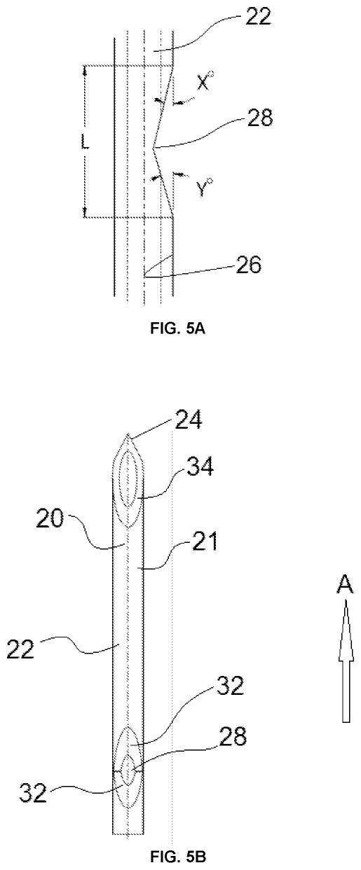

Referring to another embodiment shown in FIGS. 5A & 5B, the side and front views of a distal part of the needle respectively with at least one opening or hole 28 is shown. This embodiment differs from the previous embodiment in that the opening 28 in the needle shaft 22 is formed by two slanted cuts 32. The cuts 32 have a meeting point at the middle of the length L. One of the cuts 32 has an angle X increasing in value in the direction opposite to the needle tip 24 towards proximal end 18 of the needle 21. Whereas the second cut 32 has an angle Y increasing in value in the direction of the needle tip 24 towards distal end 20 of the needle 21. The slant angles with or without radius R defined by X and Y in FIG. 5A can vary and be within the range of 0° to 89°.

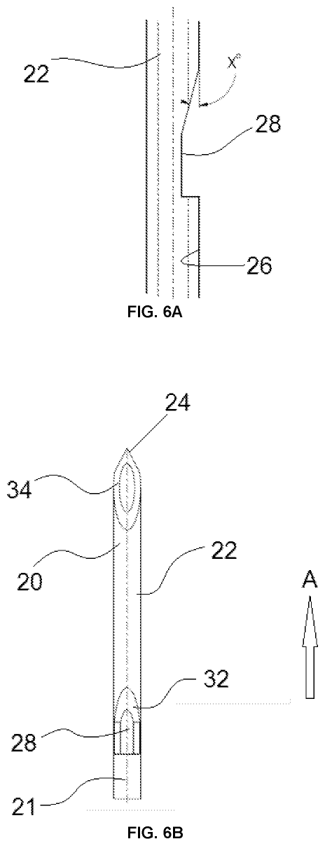

Referring now to FIGS. 6A and 6B, the side and front views of a distal part of the needle 21 respectively with at least one opening 28 according to yet another embodiment of the present invention is illustrated. As can be seen, the opening or hole 28 has at least one slanted cut 32 at an angle increasing in value opposite to the direction of the needle tip 24 toward proximal end 18 of the needle 21. The slant angle with or without radius R defined by X in FIG. 6A can vary and be within the range of 0° to 89°.

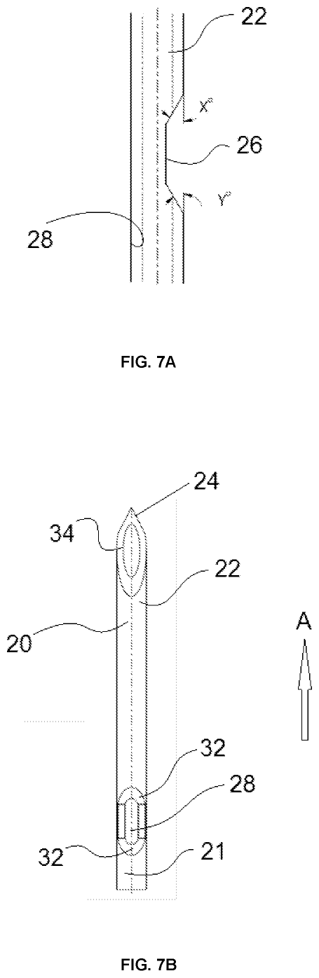

Referring to another embodiment shown in FIGS. 7A & 7B, the side and front views of a distal part of the needle 21 respectively with at least one opening or hole 28 is shown. This embodiment differs from the previous embodiment in that the opening or hole 28 in the needle shaft 22 is formed by two slanted cuts 32. One of the cuts 32 has an angle X increasing in value in the direction opposite to the needle tip 24 toward proximal end 18 of the needle 21. Whereas the second cut 32 has an angle Y increasing in value in the direction of the needle tip 24 towards distal end 20 of the needle 21. The two cuts 32 have a meeting point with an area in between which may be straight or angled. The slant angles with or without R defined by X and Y in FIG. 7A can vary and be within the range of 0° to 89°.

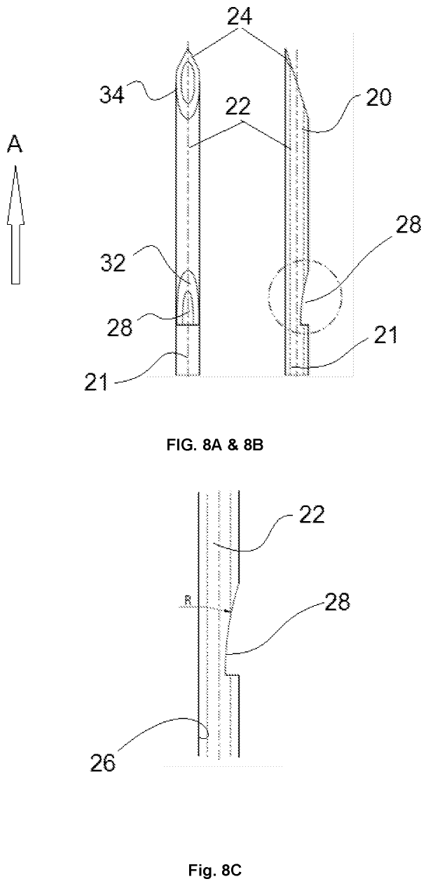

Referring to yet another embodiment shown in FIGS. 8A & 8B, the front and side views of a distal part of the needle 21 respectively with at least one opening or hole 28 is shown. FIG. 8C illustrates a detailed view of the encircled portion of FIG. 8B. As can be seen, the opening 28 has at least one slanted radial cut 32. The radius of the slanted cut 32 defined by R in FIG. 8C can have varying length.

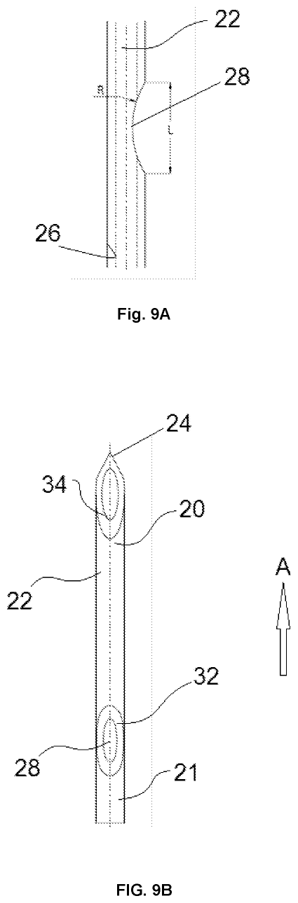

FIGS. 9A & 9B illustrate side and front views of a distal part of the needle 21 respectively with at least one opening or hole 28 according to another embodiment of the present invention. As can be seen, the opening 28 has at least one slanted radial cut 32. The radius of the slanted cut is defined by R in FIG. 9A and the radius R of the cut 32 can have varying length.

In each of the embodiments discussed above, the needle shaft 22 has a generally constant principal profile, except for at least one engagement means 36 in the form of a change in profile or enlargement as shown in FIG. 1B which can be provided along an axial position proximal or distal to the opening 28. In one embodiment, the opening 28 can be provided in between the engagement mean 36 and the needle tip 24. In an alternative embodiment, the engagement means 36 can be provided in between the needle tip 24 and the opening 28. Preferably, the engagement means 36 is made by crimping of the needle 21. However, it could also be made by welding, milling, cold heading or expanding of the needle 21.

Referring now to FIGS. 12 and 13, one implementation of the needle guard 38 of the intravenous catheter apparatus 10 as also shown in FIG. 1 is illustrated. The needle guard 38 comprises a base portion 40 and first 42 and second 44 arms extending from a distal side of the base portion 40 generally in the axial direction A. The base portion 40 and the arms 42, 44 are integrally made of a plastic material, for example by injection molding. In its ready position the needle 21 extends completely through the needle guard 38.

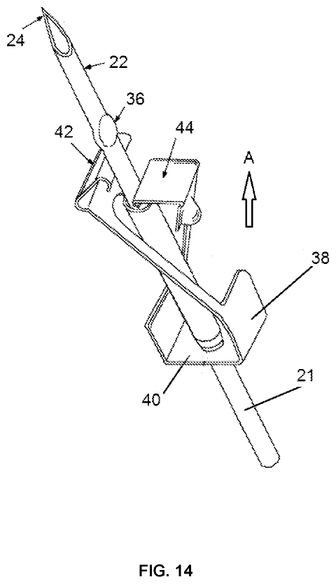

Referring now to FIG. 14, another implementation of the needle guard 38 which can be used with the intravenous catheter apparatus 10 is illustrated. The needle guard 38 comprises a base portion 40 and first 42 and second 44 arms extending from a distal side of the base portion 40 generally in the axial direction A.

The described needle 21 with the opening can be be used with any suitable intravenous system or device. In one example, the needle may be used with any suitable catheter assembly, including an over-the-needle peripheral IV catheter assembly. Thus, the needle 21 with the opening 28 as described above can be used with all the medical devices which contains and has usages of a needle 21.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise.

It will be further understood that the terms “comprises” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof Unless otherwise defined, all terms (including technical and scientific terms) used herein have the same meaning as commonly understood by one of ordinary skill in the art to which this present disclosure belongs. It will be further understood that terms used herein should be interpreted as having a meaning that is consistent with their meaning in the context of this specification and the relevant art and will not be interpreted in an idealized or overly formal sense unless expressly so defined herein.

### LIST OF REFERENCE NUMERALS:

10 intravenous catheter apparatus

12 catheter hub

14 catheter tube/catheter

16 needle hub

18 proximal end

20 distal end

21 needle

22 needle shaft

24 needle tip

26 lumen

28 opening or hole

30 space

32 slanted cut/slanted radial cut

34 bevel

36 engagement means

38 needle guard

40 base portion

42 first arm

44 second arm

46 through bore

A Axial direction

L length

W width

R radius

1. An intravenous catheter apparatus comprising:
a tubular catheter having a proximal end and a distal end mounted to a catheter hub;
a needle defining an axial direction (A) has a wall defining a lumen, a bevel and having a needle shaft with a needle tip at a distal end and a needle hub mounted to the proximal end of the needle shaft;
a needle guard arranged movably on the needle shaft;
wherein said needle shaft extends through said tubular catheter such that said needle of said needle protrudes from said distal end of said tubular catheter;
wherein said needle is provided with an engagement means adapted to engage with said needle guard in order to prevent said needle guard from sliding off said needle tip; and
wherein said needle has at least one opening having at least one slanted cut.

2. The catheter apparatus as claimed in claim 1, wherein the opening has a slanted cut at an angle increasing in value opposite to the direction of the needle tip towards proximal end of the needle.

3. The catheter apparatus as claimed in claim 1, wherein the opening has at least one slanted cut at an angle increasing in value in the direction of the needle tip towards distal end of the needle.

4. The catheter apparatus as claimed in claim 1, wherein the opening is formed by two slanted cuts in that one of the cuts has a first angle increasing in value in the direction opposite to the needle tip towards proximal end of the needle and the second cut has a second angle increasing in value in the direction of the needle tip towards distal end of the needle.

5. The catheter apparatus as claimed in claim 1, wherein the first angle and the second angle of the slant is within the range of 0° to 89°.

6. The catheter apparatus as claimed in claim 1, wherein the opening has a length and a width of varying dimensions.

7. The catheter apparatus as claimed in claim 1, wherein opening has at least one slanted radial cut having a radius wherein the radius of the slanted cut has varying length.

8. The catheter apparatus as claimed in claim 4, wherein the two cuts have a meeting point at the middle of the length.

9. The catheter apparatus as claimed in claim 4, wherein the two cuts have a meeting point with an area in between which may be straight or angled.

10. The catheter apparatus as claimed in claim 7, wherein the opening is formed by two slanted cuts in that one of the cuts has a first angle increasing in value in the direction opposite to the needle tip towards proximal end of the needle and the second cut has a second angle increasing in value in the direction of the needle tip towards distal end of the needle, wherein the first angle and the second angle with or without radius of the slant is within the range of 0° to 89°.

11. The catheter apparatus as claimed in claim 1, wherein the slanted cut of the opening has a shape selected from the group consisting of oval, elliptical, square, rectangular, triangular, circular, polygonal, and irregular.

12. The catheter apparatus as claimed in claim 1, wherein the opening can be positioned on the needle shaft so as to face in a direction that is substantially similar to, opposite to, perpendicular to, or otherwise offset from the direction in which the bevel of the needle faces.

13. The catheter apparatus as claimed in claim 1, wherein said opening is provided closer to the needle tip.

14. The catheter apparatus as claimed in claim 1, wherein the distance between the needle tip and the opening is arranged such that the opening is covered by the catheter tube.

15. The catheter apparatus as claimed in claim 1, wherein the opening can be positioned anywhere from just proximal of the needle tip to just distal of the catheter hub.

16. The catheter apparatus as claimed in claim 1, wherein the opening is provided in between the engagement means and the needle tip.

17. The catheter apparatus as claimed in claim 1, wherein the engagement means is provided in between the needle tip and the opening.

18. The catheter apparatus as claimed in claim 1, wherein the opening has two sides in that one of the sides is straight and the other side is angled.

19. The catheter apparatus as claimed in claim 1, wherein the opening or hole provided anywhere on the circumference of the needle shaft in a 360° position except of the bottom of the needle shaft in a direction opposite to the bevel of the needle.

20. The catheter apparatus as claimed in claim 1, wherein the opening or hole has a shape like a perforation forming an opening of a size being enough to allow a blood flashback therethrough.

21. The catheter apparatus as claimed in claim 1, the needle shaft comprises at least one aperture on the needle shaft.
